(19) United States
(12) Patent Application Publication
(10) Pub. No.: US 2018/0116576 A1
(43) Pub. Date: May 3, 2018

(54) MULTIPORT BLOOD COLLECTION DEVICE

(71) Applicant: S2 MedTech, LLC, Midland, TX (US)

(72) Inventors: Stanley T. Weiner, Midland, TX (US); John B. Messer, JR., Jarrell, TX (US); Brandy May Marshall, Hutto, TX (US)

(21) Appl. No.: 15/335,604

(22) Filed: Oct. 27, 2016

(51) Int. Cl. A61B 5/155; A61B 5/15

(52) U.S. Cl. CPC A61B 5/155; A61B 5/15003; A61B 5/150221; A61B 5/15074; A61B 5/150732

(57) ABSTRACT

A multiport blood collection device includes a rearward retaining portion including three couplers, each configured to receive a respective blood sample container, a forward adapter portion configured to receive a connector through which tubing coupled to a hypodermic needle is coupled to the device, and a branched passageway within the device to simultaneously divert blood extracted by the hypodermic needle from the tubing through the connector to respective blood sample containers inserted in each of the couplers. The paths through the multiport blood collection device to each of the couplers are coplanar, are symmetric about the device body, and have the same diameter. The path diameter is greater than and proportional to the inner diameter of the hypodermic needle. Each path includes at most one turn, the turn being at an angle less than ninety degrees. A portion of the branched passageway may be formed by a multi-position valve.

### BACKGROUND

### Field of the Disclosure

The present disclosure relates to blood collection systems, and more specifically, to a multiport blood collection device.

### Description of the Related Art

In many, if not the majority, of cases, when blood samples are drawn from a patient for testing purposes, multiple vials of blood are required, with the most common number of vials required being three. In some cases, the vials into which blood is drawn for different tests include different culture media, sample preservation material, or another material. The collection of blood samples is often performed using an evacuated blood collection system. In such systems, one end of a blood collection needle protruding from an evacuated blood collection tube holder is inserted into the vein of a patient from which blood is to be drawn. Subsequently, successive evacuated blood collection tubes are coupled to the evacuated blood collection tube holder to collect multiple blood samples. As each of the evacuated blood collection tubes is coupled to the evacuated blood collection tube holder, the other end of the blood collection needle pierces a cover over the forward end of the evacuated blood collection tube. The difference in pressure created when the cover is pierced causes blood to flow into the evacuated blood collection tube.

The discomfort experienced by the patient is related to the time that the hypodermic needle is inserted in the vein. This time is extended when a phlebotomist swaps out one vial for another. In addition, the opportunity for movement of the hypodermic needle and for bruising caused by such movement is increased as the number of vials of blood drawn increases. In some cases, while collecting multiple blood samples in multiple vials, the phlebotomist may become distracted, causing their attention to the position of the hypodermic needle to lag. This may result in the removal and subsequent reinsertion of the hypodermic needle, further adding to the discomfort of the patient.

### SUMMARY

The disclosure provides a multiport blood collection device including a rearward retaining portion including three couplers, each configured to receive a respective blood sample container, a forward adapter portion configured to receive a connector through which a piece of tubing that is coupled to a hypodermic needle on one end is coupled to the device on the opposite end, and a branched passageway within the device to simultaneously divert blood extracted by the hypodermic needle from a piece of tubing coupled to the device through the connector inserted in the adapter portion to respective blood sample containers inserted in each of the couplers.

A first portion of the branched passageway diverts blood from the tubing to a branch point within the device, a center line of the first portion of the branched passageway being coplanar with and aligned to a center line of the adapter portion.

A second portion of the branched passageway diverts blood from the branch point to a first one of the couplers. A center line of the second portion of the branched passageway is coplanar with the center line of the first portion of the branched passageway. The center line of the second portion of the branched passageway is aligned to the center line of the first portion of the branched passageway and to a center line of the first coupler.

A third portion of the branched passageway diverts blood from the branch point to a second one of the couplers. A center line of the third portion of the branched passageway is coplanar with the center line of the first portion of the branched passageway. The center line of the third portion of the branched passageway is aligned to a center line of the second coupler. The center line of the third portion of the branched passageway branches away from the second portion of the branched passageway on a first side of the second portion of the branched passageway at a fixed angle that is less than ninety degrees.

A fourth portion of the branched passageway diverts blood from the branch point to a third one of the couplers. A center line of the fourth portion of the branched passageway is coplanar with the center line of the first portion of the branched passageway. The center line of the fourth portion of the branched passageway is aligned to a center line of the third coupler. The center line of the fourth portion of the branched passageway branches away from the second portion of the branched passageway on a second side of the second portion of the branched passageway at a fixed angle that is less than ninety degrees.

In any of the disclosed embodiments, the connector may include a female Luer taper fitting and the adapter may include a male Luer taper fitting.

In any of the disclosed embodiments, each of the couplers may include a female Luer taper fitting.

In any of the disclosed embodiments, the multiport blood collection device may be comprised of a single solid body.

In any of the disclosed embodiments, the multiport blood collection device may be fashioned from a polycarbonate, polyetherimide, or nylon material.

In any of the disclosed embodiments, the multiport blood collection device may be fashioned from a material that is chemically resistant, that is transparent, and that has smoothness between ten and twenty microns.

In any of the disclosed embodiments, the multiport blood collection device may be disposable.

In any of the disclosed embodiments, the fixed angle at which the center line of the third portion of the branched passageway branches away from the second portion of the branched passageway and the fixed angle at which the center line of the fourth portion of the branched passageway branches away from the second portion of the branched passageway may be substantially equal.

In any of the disclosed embodiments, the fixed angle at which the center line of the third portion of the branched passageway branches away from the second portion of the branched passageway may be about fifty degrees.

In any of the disclosed embodiments, the respective distances from the branch point to each of the couplers along the second, third, and fourth portions of the branched passageways may be substantially equal.

In any of the disclosed embodiments, each of the blood sample containers may include an evacuated blood collection tube within an evacuated blood collection tube holder to which a blood collection needle is coupled. The blood collection needle may extend into the portion of the branched passageway that diverts blood to the coupler into which the blood sample container is inserted.

In any of the disclosed embodiments, the diameters of the first, second, third, and fourth portions of the branched passageway may be substantially equal.

In any of the disclosed embodiments, the diameter of each of the first, second, third, and fourth portions of the branched passageway may be directly proportional to an inside diameter of the hypodermic needle.

In any of the disclosed embodiments, a ratio between the diameter of each of the first, second, third, and fourth portions of the branched passageway and the inside diameter of the hypodermic needle may be between 3:1 and 5:1.

In any of the disclosed embodiments, the multiport blood collection device may further include a bracket through which a flexible strap is to be inserted. The strap may include a fastener to attach the multiport blood collection device to an arm during use.

In any of the disclosed embodiments, the multiport blood collection device may further include a multi-position valve, and a portion of the branched passageway, including the branch point, may be located within the multi-position valve. The branched passageway may simultaneously divert blood from a piece of tubing coupled to the device to respective blood sample containers inserted in each of the three couplers when the multi-position valve is in a first position. The branched passageway may divert blood from a piece of tubing coupled to the device to respective blood sample containers inserted in fewer than three couplers when the multi-position valve is in a second position. The multi-position valve may prevent blood from being diverted from a piece of tubing coupled to the three couplers when the multi-position valve is in a third position.

The disclosure also provides a blood collection system including a multiport blood collection device, a hypodermic needle, a connector, and a piece of tubing coupled to the hypodermic needle on one end and coupled to the connector on the opposite end. The multiport blood collection device includes a rearward retaining portion including three couplers, each configured to receive a respective blood sample container, a forward adapter portion configured to receive the connector, and a branched passageway within the device to simultaneously divert blood extracted by the hypodermic needle from a piece of tubing coupled to the device through the connector inserted in the adapter portion to respective blood sample containers inserted in each of the couplers.

A first portion of the branched passageway diverts blood from the tubing to a branch point within the device, a center line of the first portion of the branched passageway being coplanar with and aligned to a center line of the adapter portion.

A second portion of the branched passageway diverts blood from the branch point to a first one of the couplers. A center line of the second portion of the branched passageway is coplanar with the center line of the first portion of the branched passageway. The center line of the second portion of the branched passageway is aligned to the center line of the first portion of the branched passageway and to a center line of the first coupler.

A third portion of the branched passageway diverts blood from the branch point to a second one of the couplers. A center line of the third portion of the branched passageway is coplanar with the center line of the first portion of the branched passageway. The center line of the third portion of the branched passageway is aligned to a center line of the second coupler. The center line of the third portion of the branched passageway branches away from the second portion of the branched passageway on a first side of the second portion of the branched passageway at a fixed angle that is less than ninety degrees.

A fourth portion of the branched passageway diverts blood from the branch point to a third one of the couplers. A center line of the fourth portion of the branched passageway is coplanar with the center line of the first portion of the branched passageway. The center line of the fourth portion of the branched passageway is aligned to a center line of the third coupler. The center line of the fourth portion of the branched passageway branches away from the second portion of the branched passageway on a second side of the second portion of the branched passageway at the fixed angle.

The disclosure also provides a method for collecting multiple blood samples, the method including placing a plurality of evacuated blood collection tubes, each positioned within an evacuated blood collection tube holder, into respective couplers on a rearward portion of a multiport blood collection device. The multiport blood collection device includes a branched passageway configured to divert blood from to an adapter on a forward portion of the multiport blood collection device to each of the evacuated blood collection tubes simultaneously along a respective path containing at most one angle, where the angle, if any, is less than ninety degrees.

The method also includes coupling a connector of a winged infusion set to the adapter, inserting a hypodermic needle of the winged infusion set into a vein from which blood is to be drawn, pushing the evacuated blood collection tubes forward into their respective holders to begin drawing blood into the evacuated blood collection tubes, removing the hypodermic needle from the vein subsequent to the evacuated blood collection tubes being filled, removing the evacuated blood collection tubes from the multiport blood collection device, and disposing of the multiport blood collection device following a single use.

### BRIEF DESCRIPTION OF THE DRAWINGS

For a more complete understanding of the present invention and its features and advantages, reference is now made to the following description, taken in conjunction with the accompanying drawings, which represent one or more embodiments of the disclosure, which maybe combined with one another unless clearly mutually exclusive, and in which:

FIG. 1 is a schematic diagram of a blood collection system while the system is in use, in accordance with some embodiments of the present disclosure;

FIG. 2A is a top-view cross-sectional diagram of a multiport blood collection device, in accordance with some embodiments of the present disclosure;

FIG. 2B is a perspective view of the exterior of the multiport blood collection device of FIG. 2A, in accordance with some embodiments of the present disclosure;

FIG. 3A is a top-view cross-sectional diagram of a multiport blood collection device with a bracket through which a flexible strap may be inserted, in accordance with some embodiments of the present disclosure;

FIG. 3B is a perspective view of the exterior of the multiport blood collection device of FIG. 3A, in accordance with some embodiments of the present disclosure;

FIG. 3C is a side-view cross-sectional diagram of the multiport blood collection device of FIG. 3A, in accordance with some embodiments of the present disclosure;

FIG. 4 is a perspective view illustrates an embodiment of a multiport blood collection device that includes integrated blood sample container holders;

FIG. 5A is a top-view cross-sectional diagram of a multiport blood collection device containing a multi-position valve, in accordance with some embodiments of the present disclosure;

FIG. 5B is a perspective view of disassembled components of the multiport blood collection device of FIG. 5A;

FIGS. 6A through 6E depict a series of top-view cross-sectional schematic diagrams of a multi-position valve through which a branched passageway is formed in different positions, in accordance with some embodiments of the present disclosure; and

FIG. 7 is a flow diagram of a method for collecting blood samples using a multiport blood collection device, in accordance with some embodiments of the present disclosure.

### DETAILED DESCRIPTION

In the following description, details are set forth by way of example to facilitate discussion of the disclosed subject matter. It should be apparent to a person of ordinary skill in the field, however, that the disclosed embodiments are exemplary and not exhaustive of all possible embodiments. In addition, elements of various embodiments may be combined with one another unless clearly mutually exclusive.

In at least some embodiments of the present disclosure, a multiport blood collection device includes multiple couplers, each configured to receive a respective blood sample container, an adapter configured to receive a connector through which tubing coupled to a hypodermic needle on one end is coupled to the device on the other end, and a branched passageway within the device to simultaneously divert blood extracted by the hypodermic needle from the tubing through the connector to respective blood sample containers inserted in each of the couplers. By allowing multiple blood samples to be taken simultaneously using a single insertion of a single hypodermic needle, both the time to perform the collection of multiple blood samples and the discomfort of the patient may be reduced when the multiport blood collection device is employed.

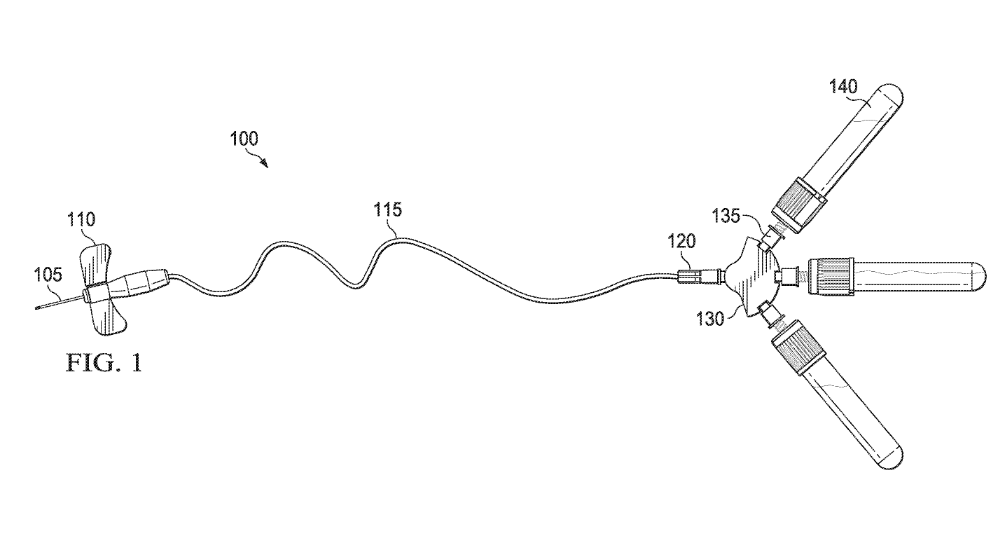

FIG. 1 is a schematic diagram a blood collection system 100 while the system is in use, in accordance with some embodiments of the present disclosure. In this example embodiment, blood collection system 100 includes a multiport blood collection device 130, a hypodermic needle 105, a bilateral wing assembly 110, a connector 120, and a piece of tubing 115 coupled to the hypodermic needle 110 on one end and coupled to the connector 120 on the opposite end. In this example embodiment, the hypodermic needle 105, bilateral wing assembly 110, connector 120, and the piece of tubing 155 form a winged infusion set. A winged infusion set is sometimes referred to as a “butterfly” or “scalp vein” set.

In this example embodiment, multiport blood collection device 130 is configured to receive, through an adapter on a forward portion of the device, connector 120. Multiport blood collection device 130 is configured to receive multiple blood sample container adapters 135 through respective couplers on a rearward portion of the device. Multiport blood collection device 130 may include, within the device, a branched passageway configured to simultaneously divert blood extracted by the hypodermic needle 105 and received by multiport blood collection device 130 through tubing 115 and connector 120 to respective blood sample containers 140 coupled to multiport blood collection device 130 through respective blood sample container adapters 135. In some embodiments, each of the blood sample containers 140 may be of the same size and type. In other embodiments, one or more of the blood sample containers 140 may include a culture media, a sample preservation material, or another material and the culture media, sample preservation material, or another material included in two or more of the blood sample containers 140 may be of different types. In some embodiments, one or more of the blood sample containers 140 may include an anti-coagulant. Thus, using multiport blood collection device 130, multiple blood samples may be collected simultaneously for different purposes and may be diverted to respective blood sample containers 140 containing different materials without cross-contamination.

During use of the multiport blood collection device 130, each of the blood sample container adapters 135 may hold a respective blood sample container 140. In at least some embodiments, the connector 120 may include a female Luer taper fitting and the adapter on the forward portion of the device (not shown) may include a male Luer taper fitting. In at least some embodiments, each of the blood sample container adapters 135 may include a female Luer taper fitting. In at least some embodiments, each of the blood sample containers 140 may be an evacuated blood collection tube and each of the blood sample container adapters 135 may be an evacuated blood collection tube holder to which a blood collection needle is coupled. When the evacuated blood collection tube and evacuated blood collection tube holder are positioned for use in multiport blood collection device 130, a forward portion of the blood collection needle of the evacuated blood collection tube holder may extend into the portion of the branched passageway that diverts blood toward the evacuated blood collection tube holder into which the evacuated blood collection tube is inserted.

During use of the blood collection system 100, once hypodermic needle 105 is inserted into a vein and the evacuated blood collection tubes are pushed down into their respective evacuated blood collection tube holders, a rearward portion of the blood collection needle of each evacuated blood collection tube holder may pierce a rubber cover over the forward end of the evacuated blood collection tube inserted in the holder, and the suction created by the pressure difference may cause blood to begin flowing in the blood collection system 100. In the device as shown in FIG. 1, blood may flow into hypodermic needle 105 and from hypodermic needle 105 to multiport blood collection device 130 through tubing 115 and connector 120. The branched passageway within multiport blood collection device 130 may divert the blood to all of the blood sample containers 140 simultaneously. For example, at a point in time depicted in FIG. 1, each of blood sample containers 140 contains an equal amount of blood.

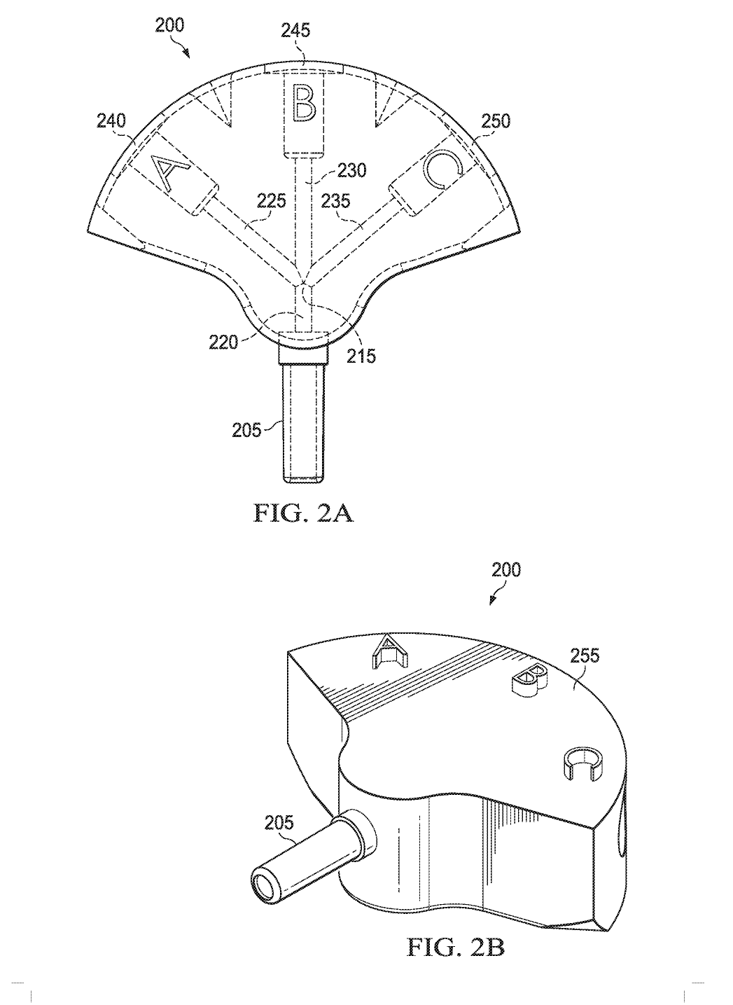

FIGS. 2A and 2B illustrate different views of an embodiment of a multiport blood collection device 200. FIG. 2A depicts a cross-section of multiport blood collection device 200 from the top of the device, with the rearward portion of the device at the top of FIG. 2A and the forward portion of the device at the bottom of FIG. 2A. In this example embodiment, multiport blood collection device 200 includes an adapter 205 through which a connector on one end of a piece of tubing couples the piece of tubing to the multiport blood collection device 200. In at least some embodiments, the connector may include a female Luer taper fitting and the adapter may include a male Luer taper fitting.

In FIG. 2A, a branched passageway within multiport blood collection device 200 is shown using dashed lines. The branched passageway includes a first portion 220 from adapter 205, or from a piece of tubing coupled thereto, to branch point 215. The branched passageway also includes a second portion 230 from branch point 215 to coupler B (245), a third portion 225 from branch point 215 to coupler A (240), and a fourth portion 235 from branch point 215 to coupler C (250). Each of the couplers 240, 245, and 250 is configured to receive a blood sample container. For example, in at least some embodiments, each of the couplers 240, 245, and 250 includes a female Luer taper fitting in which to receive an evacuated blood collection tube.

In the example embodiment illustrated in FIG. 2A, a center line of the second portion 230 of the branched passageway is coplanar with and aligned to a center line of the first portion 220 and a center line of adapter 205. The third portion 225 and the fourth portion 235 of the branched passageway are symmetric about a center line passing through the body of multiport blood collection device 200 and about the second portion 230. Each of the third portion 225 and the fourth portion 235 of the branched passageway branches off of the first portion 220 at the same angle, which is less than 90°. A branched passageway that includes multiple turns or that includes turns at angles of 90° or greater can restrict the flow of blood through a multiport blood collection device to an unsatisfactory degree. In addition, if the diameter of the branched passageway is not sufficiently larger than the inner diameter of the hypodermic needle through which the blood is drawn, this may further restrict the flow of blood through a multiport blood collection device. In some cases, even if the flow of blood is not restricted to a degree that the blood sample containers cannot be filled, the pressure caused by a restriction in blood flow may cause hemolysis of the blood and reduce its suitability for certain blood tests.

In one embodiment, the angle at which each of these portions of the branched passageway branches off of the first portion 220, as measured from center lines of each of the portions of the branched passageway, is approximately 50°.

As shown in FIG. 2A, the center lines of third portion 225 and fourth portion 235 may also be coplanar with a center line of the first portion 220, a center line of the second portion 230, and a center line of adapter 205. The center line of second portion 230 is aligned to a center line of coupler B (245), the center line of third portion 225 is aligned to a center line of coupler B (240), and the center line of fourth portion 235 is aligned to a center line of coupler C (250).

In at least some embodiments, the respective distances from the branch point 215 to each of the couplers along the second, third, and fourth portions of the branched passageways are substantially equal. This may allow the blood sample containers inserted in each of the couplers to fill at approximately the same rate. In at least some embodiments, the diameters of the first, second, third, and fourth portions of the branched passageway are also substantially equal. In some embodiments, the diameter of each of the first, second, third, and fourth portions of the branched passageway is directly proportional to an inside diameter of the hypodermic needle used to draw the blood to be diverted along the branched passageway from a patient. In one embodiment, the ratio between the diameter of each of the first, second, third, and fourth portions of the branched passageway and the inside diameter of the hypodermic needle may be between 3:1 and 5:1. For example, in one embodiment, the ratio may be approximately 4.63:1.

FIG. 2B illustrates a perspective view of multiport blood collection device 200. Visible in FIG. 2B are the body of multiport blood collection device 200, shown as 255, and adapter 205, which protrudes from the forward portion of multiport blood collection device 200. In at least some embodiments, multiport blood collection device 200 may form a single solid body. In various embodiments, the multiport blood collection device 200 may be fashioned from any of a variety of materials that are approved by the U.S. Food and Drug Administration (FDA) for use in medical devices. In some embodiments, the multiport blood collection device 200 may be fashioned from a material that is chemically resistant. In some embodiments, the multiport blood collection device 200 may also be fashioned from a material that is transparent, allowing a phlebotomist to monitor the rate at which the blood sample containers are filled through observation. In some embodiments, the multiport blood collection device 200 may be fashioned from a material having a smoothness of between ten and twenty microns. This may further reduce the change that the blood flowing through the device is subjected to hemolysis. In some embodiments, the multiport blood collection device 200 may be fashioned from an FDA approved plastic, such as a polycarbonate, polyetherimide, or nylon material. In at least some embodiments, the multiport blood collection device 200 may be disposable. For example, the device may be fashioned from relatively inexpensive materials using a relatively inexpensive process, making it suitable for use as a single-use item.

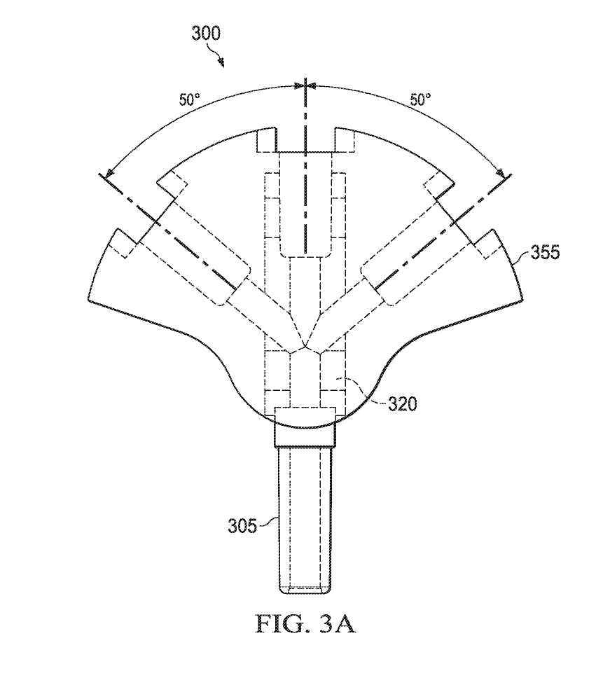

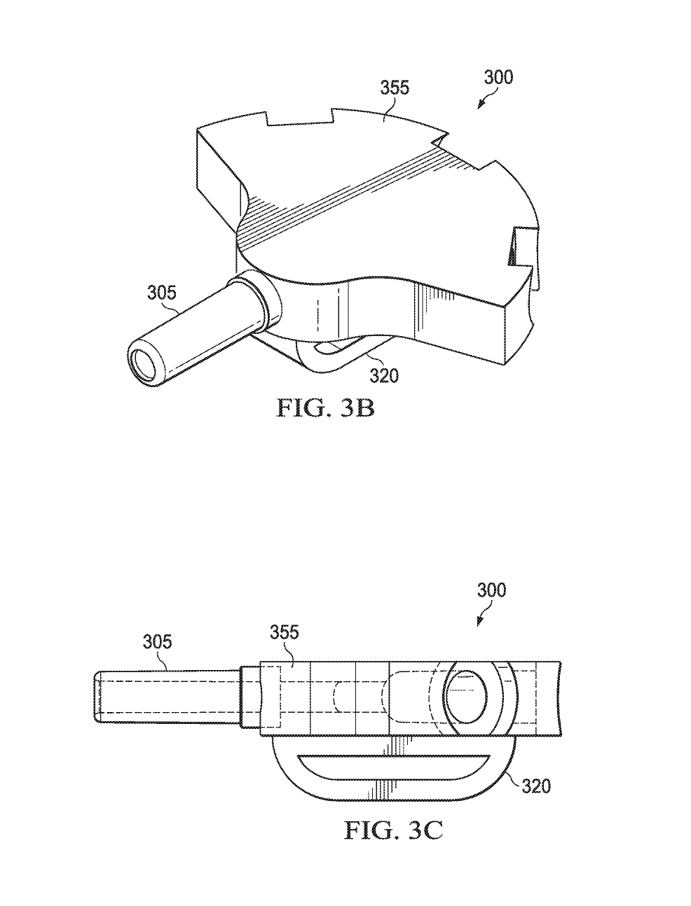

In some embodiments of the present disclosure, the multiport blood collection device 200 may include a mechanism for securing the device to the patient during a blood draw. FIGS. 3A through 3C illustrate different views of a multiport blood collection device 300 that includes a bracket through which a flexible strap is to be inserted. For example, FIG. 3A depicts a cross-section of multiport blood collection device 300 from the top of the device, with the rearward portion of the device at the top of FIG. 3A and the forward portion of the device at the bottom of FIG. 3A. As illustrated in FIG. 3A, multiport blood collection device 300 includes a body 355, and an adapter 305, which protrudes from the forward portion of multiport blood collection device 300. Within the body 355, a branched passageway is configured to divert blood received through adapter 305 to blood sample containers inserted into each of three couplers on the rearward portion of the multiport blood collection device 300.

As illustrated in this example embodiment, the angle at which each of the two side portions of the branched passageway branches off of the central portion of the branched passageway, as measured from center lines of each of the portions of the branched passageway, is approximately 50°.

In this example embodiment, multiport blood collection device 300 includes a bracket 320 through which a flexible strap is to be inserted. The strap may include a fastener to attach the multiport blood collection device to an arm from which blood is being drawn during use. In various embodiments, the strap may be made of any suitable fabric and may include one or more snaps or hook-and-eye closures with which to secure the strap in place after it is wrapped around the arm. In other embodiments, the strap may be made of any suitable fabric and may include a hook and loop fastener. In still other embodiments, the strap may be similar to a “slap bracelet” that includes layered, flexible stainless steel bi-stable spring bands sealed within a fabric, silicone, or plastic cover. In these and other embodiments, the flexible strap may allow multiport blood collection device 300 to be secured to arms of different sizes in order to hold the multiport blood collection device 300 relatively still while blood is drawn.

FIG. 3B illustrates a perspective view of multiport blood collection device 300. Visible in FIG. 3B are the body of multiport blood collection device 300, shown as 355, adapter 305, which protrudes from the forward portion of multiport blood collection device 300, and bracket 320, which protrudes from the underside of multiport blood collection device 300. FIG. 3C illustrates a side view of multiport blood collection device 300. Visible in FIG. 3C are the body of multiport blood collection device 300, shown as 355, adapter 305, which protrudes from the forward portion of multiport blood collection device 300, and bracket 320, which protrudes from the underside of multiport blood collection device 300.

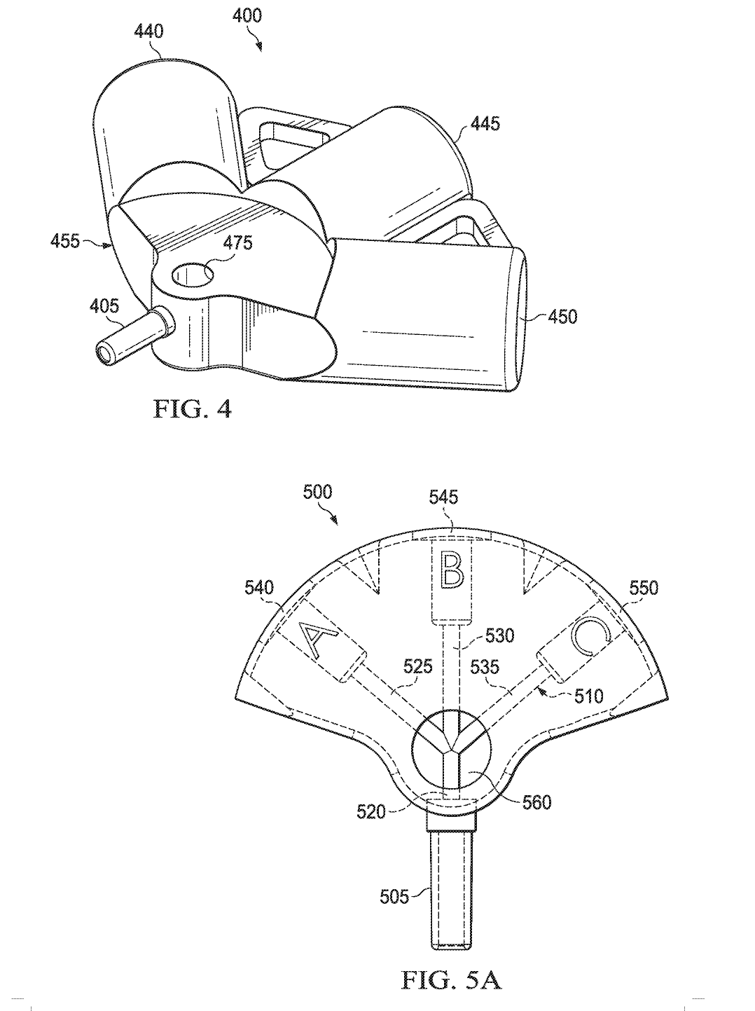

FIG. 4 illustrates an embodiment of a multiport blood collection device 400 that includes integrated blood sample container holders. More specifically, FIG. 4 illustrates a perspective view of a multiport blood collection device 400 into which blood sample container holders are integrated into the body of the device. Visible in FIG. 4 are the body of multiport blood collection device 400, shown as 455, adapter 405, which protrudes from the forward portion of multiport blood collection device 400, and three integrated blood sample container holders 440, 445, and 450 on the rearward portion of multiport blood collection device 400. In one embodiment, integrated blood sample container holders 440, 445, and 450 may be evacuated blood collection tube holders, each containing a blood collection needle to pierce a rubber cover over the forward end of an evacuated blood collection tube inserted in the holder during use of the multiport blood collection device 400.

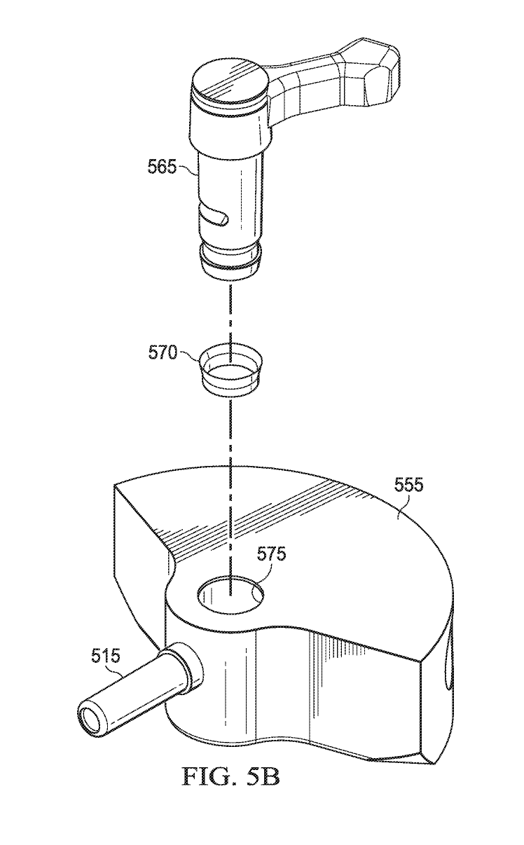

In the example embodiment illustrated in FIG. 4, the body 455 of multiport blood collection device 400 also includes an opening 475 through which a valve stem of a multi-position valve within multiport blood collection device 400 may be inserted. A multi-position valve and corresponding valve stem are illustrated in FIGS. 5A and 5B and described below, according to one embodiment.

FIGS. 5A and 5B illustrate different views of an embodiment of a multiport blood collection device 500 containing a multi-position valve 560. For example, FIG. 5A depicts a cross-section of multiport blood collection device 500 from the top of the device, with the rearward portion of the device at the top of FIG. 5A and the forward portion of the device at the bottom of FIG. 5A. In this example embodiment, multiport blood collection device 500 includes an adapter 505 through which a connector on one end of a piece of tubing couples the piece of tubing to the multiport blood collection device 500. In at least some embodiments, the connector includes a female Luer taper fitting and the adapter includes a male Luer taper fitting.

In FIG. 5A, a branched passageway within multiport blood collection device 500 is shown using dashed lines. The branched passageway includes a first portion 520 from adapter 505, or from a piece of tubing coupled thereto, to branch point 515. The branched passageway also includes a second portion 530 from branch point 515 to coupler B (545), a third portion 525 from branch point 515 to coupler A (540), and a fourth portion 535 from branch point 515 to coupler C (550). Each of the couplers 540, 545, and 550 is configured to receive a blood sample container. For example, in at least some embodiments, each of the couplers 540, 545, and 550 includes a female Luer taper fitting in which to receive an evacuated blood collection tube.

In the example embodiment illustrated in FIG. 5A, a center line of the second portion 530 of the branched passageway is coplanar with and aligned to a center line of the first portion 520 and a center line of adapter 505. The third portion 525 and the fourth portion 535 of the branched passageway are symmetric about a center line passing through the body of multiport blood collection device 500 and about the second portion 530. Each of the third portion 525 and the fourth portion 535 of the branched passageway branches off of the first portion 520 at the same angle, which is less than 90°. In one embodiment, the angle at which each of these portions of the branched passageway branches off of the first portion 520, as measured from center lines of each of the portions of the branched passageway, is approximately 50°.

The center lines of third portion 525 and fourth portion 535 are also coplanar with a center line of the first portion 520, a center line of the second portion 530, and a center line of adapter 505. The center line of second portion 530 is aligned to a center line of coupler B (545), the center line of third portion 525 is aligned to a center line of coupler B (540), and the center line of fourth portion 535 is aligned to a center line of coupler C (550). In at least some embodiments, the respective distances from the branch point 515 to each of the couplers along the second, third, and fourth portions of the branched passageways are substantially equal. In at least some embodiments, the diameters of the first, second, third, and fourth portions of the branched passageway are substantially equal. In some embodiments, the diameter of each of the first, second, third, and fourth portions of the branched passageway is directly proportional to an inside diameter of the hypodermic needle used to draw the blood to be diverted along the branched passageway from a patient.

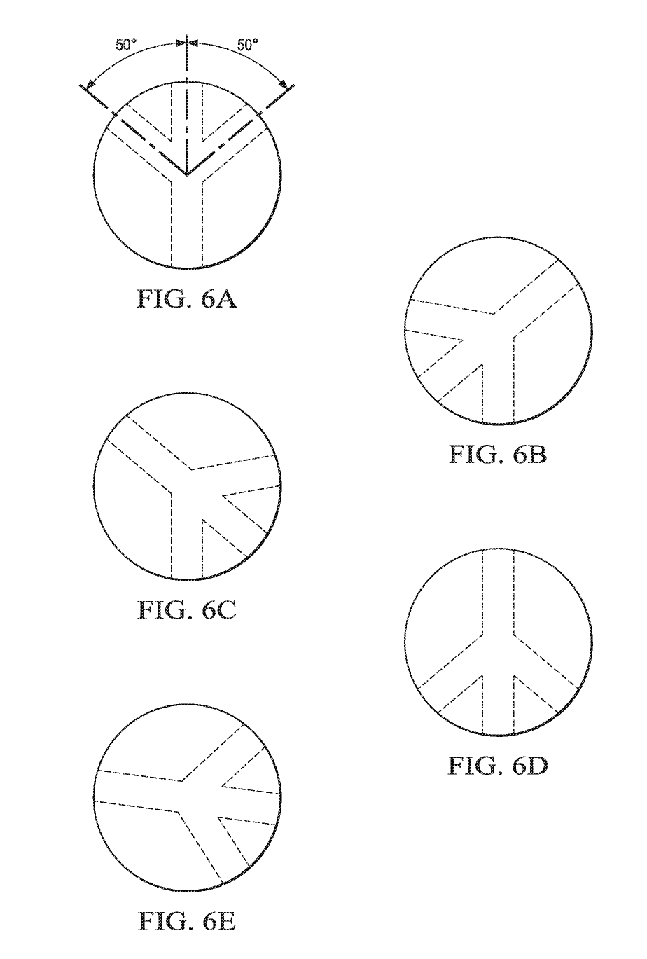

In the example embodiment illustrated in FIG. 5A, multiport blood collection device 500 includes a multi-position valve 560 for selecting which, if any, of the blood sample containers coupled to multiport blood collection device 500 are to be filled when blood is drawn using the device. A portion of the multi-position valve 560 is shown in FIG. 5A. In this example, embodiment, a portion of the branched passageway, including the branch point, is located within the multi-position valve 560. Dependent on the position of the multi-position valve 560, blood received through adapter 505 may be diverted to a blood sample container inserted in a single one of the couplers 540, 545, and 550, blood received through adapter 505 may be diverted to blood sample containers inserted in all three of the couplers 540, 545, and 550, or the multi-position valve 560 may prevent blood from flowing in a blood collection system that includes multiport blood collection device 500. Different positions of multi-position valve 560 are illustrated in FIGS. 6A through 6E and described below.

FIG. 5B illustrates multiport blood collection device 500. Visible in FIG. 5B are the body of multiport blood collection device 500, shown as 555, and adapter 505, which protrudes from the forward portion of multiport blood collection device 500. Also visible in FIG. 5B are a valve stem 565 for selecting which, if any, blood sample containers inserted in the couplers 540, 545, or 550 of multiport blood collection device 500 are to be filled during a single blood collection, and a valve seat 570 for coupling valve stem 565 to multi-position valve 560. In this example embodiment, body 555 includes an opening 575 into which valve seat 570 is placed to couple valve stem 565 to multi-position valve 560 when valve stem 565 is inserted into opening 575.

In some embodiments, a multiport blood collection device such as multiport blood collection device 500 may include a multi-position valve intended for one-time assembly and use. In such embodiments, the multiport blood collection device may have a tapered opening in the body and a tapered valve stem having a lip on the bottom may, during assembly, be forced down into the opening to snap the two pieces together. In this example embodiment, the multi-position valve may seal itself on the taper, and may not require a valve seat or an O-ring. In this example, once the valve is inserted into the multiport blood collection device, it cannot be removed without breaking it.

Each of FIGS. 6A through 6E depicts the multi-position valve 560 illustrated in FIG. 5A in a different position. For example, FIG. 6A illustrates a first position of multi-position valve 560. In this position, the first, second, third, and fourth portions of the branched passageway illustrated in FIG. 5A, shown as 520, 530, 525, and 535, respectively, are completed through multi-position valve 560. In this case, blood collected by a hypodermic needle coupled to multiport blood collection device 500 is simultaneously diverted to respective blood sample containers inserted in couplers A (540), B (545), and C (550) shown in FIG. 5A.

FIG. 6B illustrates a second position of multi-position valve 560. In this position, only the first and fourth portions of the branched passageway, shown as 520 and 535, respectively, are completed through multi-position valve 560. In this case, all of the blood collected by a hypodermic needle coupled to multiport blood collection device 500 is diverted to a blood sample container inserted in coupler C (550) shown in FIG. 5A.

FIG. 6C illustrates a third position of multi-position valve 560. In this position, only the first and third portions of the branched passageway, shown as 520 and 525, respectively, are completed through multi-position valve 560. In this case, all of the blood collected by a hypodermic needle coupled to multiport blood collection device 500 is diverted to a blood sample container inserted in coupler A (540) shown in FIG. 5A.

FIG. 6D illustrates a fourth position of multi-position valve 560. In this position, only the first and second portions of the branched passageway, shown as 520 and 530, respectively, are completed through multi-position valve 560. In this case, all of the blood collected by a hypodermic needle coupled to multiport blood collection device 500 is diverted to a blood sample container inserted in coupler B (545) shown in FIG. 5A.

FIG. 6E illustrates a fifth position of multi-position valve 560. In this position, none of the paths of the branched passageway are completed through multi-position valve 560. In this case, no blood will be collected by a hypodermic needle coupled to multiport blood collection device 500 nor will any blood be diverted to blood sample containers inserted in any of couplers A (540), B (545), or C (550) shown in FIG. 5A.

FIGS. 6A through 6E illustrate the operation of a multi-position valve through which zero, one, or three blood sample containers can be filled simultaneously using a multiport blood collection device, depending on the position of the valve. In other embodiments, a multi-position valve may also be configured to select a different number of blood sample containers to be filled simultaneously during a single blood draw. For example, in some embodiments, a multi-position valve may include positions in which two blood sample containers can be simultaneously filled during a single blood draw in addition to, or instead of, positions in which zero, one, or three blood sample containers can be filled during a single blood draw. In embodiments in which a multiport blood collection device includes fewer or greater than three couplers for receiving blood sample containers, different configurations of multi-position valves may be included in the multiport blood collection device to select which, if any, of the blood sample containers is to be filled during a given blood draw.

While several example embodiments of a multiport blood collection device are illustrated in the figures and described in detail herein, in other embodiments, a multiport blood collection device may include more, fewer, or different features than those illustrated in these example embodiments in different combinations. For example, in various embodiments, a multiport blood collection device may include any number of couplers, one or more integrated blood sample container holders, a bracket for a flexible strap, or a multi-position valve for selecting which, if any, of the blood sample containers is to be filled during a given blood draw, in any combination. In embodiments in which blood is diverted to one or both of two blood sample containers, the passageways to each of these blood sample containers through the multiport blood collection device may be coplanar and may each include at most one turn at an angle of less than 90°. Similarly, in embodiments in which blood can be diverted to any of four or more blood sample containers, the passageways to each of those blood sample containers through the multiport blood collection device may be coplanar and may each include at most one turn at an angle of less than 90°. While several example embodiments of a multiport blood collection device are illustrated in the figures and described in detail above, in other embodiments, a multiport fluid collection device similar to those described herein may be used to simultaneously collect multiple samples of other bodily fluids in appropriate sample containers. For example, blood or other types of bodily fluids drawn from a donor or patient may be diverted through a multiport fluid collection device and collected in test tubes or other types of sample containers that are coupled to the multiport fluid collection device, in different embodiments.

In at least some embodiments, a multiport blood collection device may be sized for use with hypodermic needles, tubing, or evacuated blood collection systems whose components are compliant with standards for the dimensions of these components when used for blood collection. In one embodiment in which the couplers are configured to receive blood sample container holders, a multiport blood collection device may be approximately 1.65 inches wide at its widest point, 1.65 inches long from front to back, and 0.55 inches tall. In one embodiment in which the multiport blood collection device includes integrated blood sample container holders, the device may be approximately 3.5 inches wide at its widest point, 2.75 inches long from front to back, and 0.75 inches tall at its tallest point.

In some embodiments, a multiport blood collection device such as those described herein may be provided as an element of a blood collection system. For example, a blood collection kit may include the multiport blood collection device, a hypodermic needle, a connector, and a piece of tubing coupled to the hypodermic needle on one end and coupled to the connector on the opposite end. In some embodiments, a blood collection system may also include one or more blood sample containers or blood sample container holders. In some embodiments in which the multiport blood collection device includes a bracket, the blood collection system may also include a flexible strap for securing the device to the patient during use. In at least some embodiments of a blood collection system, the entire blood collection system may be disposable.

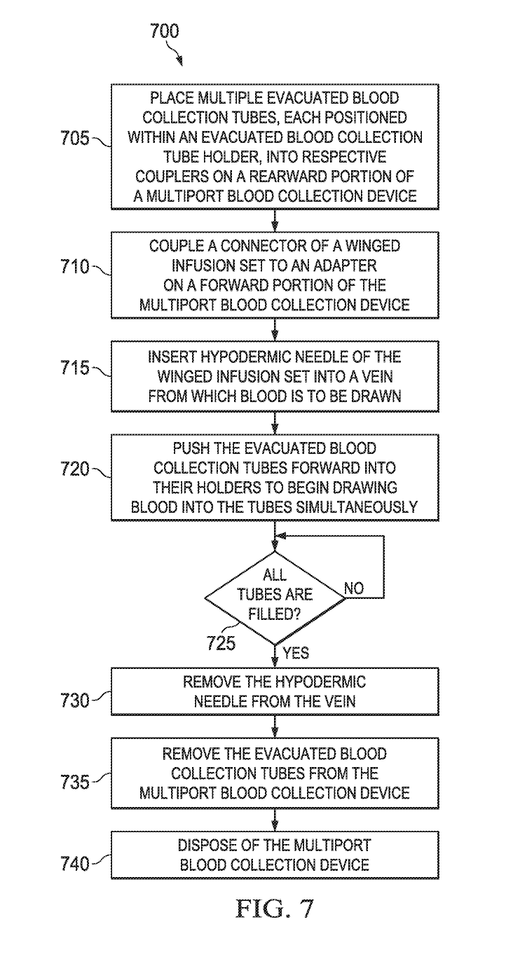

FIG. 7 is a flow diagram illustrating a method 700 for collecting blood samples using a multiport blood collection device, in accordance with some embodiments of the present disclosure. In this example embodiment, method 700 includes, at 705, placing a plurality of evacuated blood collection tubes, each positioned within an evacuated blood collection tube holder, into respective couplers on a rearward portion of a multiport blood collection device. In some embodiments, the multiport blood collection device may be similar to one of the multiport blood collection devices 130, 200, 300, 400, or 500 described herein. When initially positioned in the evacuated blood collection tube holders, the rubber covers on the evacuated blood collection tubes have not yet been pierced by the needles in the evacuated blood collection tube holders. The multiport blood collection device may include a branched passageway configured to divert blood from to an adapter on a forward portion of the multiport blood collection device to each of the plurality of evacuated blood collection tubes simultaneously along a respective path containing at most one angle, the angle being less than ninety degrees. For example, in some embodiments, one of the paths may not include any turns, while the other two paths may each include a single 50° turn.

Method 700 may include, at 710, coupling a connector of a winged infusion set to the adapter on the forward portion of the multiport blood collection device. Method 700 also includes, at 715, inserting a hypodermic needle of the winged infusion set into a vein from which blood is to be drawn. For example, the multiport blood collection device may be rested on a table or on the inner forearm of a patient that is resting on a table and the hypodermic needle may be inserted into a vein in the patient's arm. Once the hypodermic needle has been inserted, the method includes, at 720, pushing the evacuated blood collection tubes forward into their respective holders to begin drawing blood into the evacuated blood collection tubes. For example, pushing the evacuated blood collection tubes forward into their respective holders may cause the rubber covers on the evacuated blood collection tubes to be pierced by the needles in the evacuated blood collection tube holders, after which the pressure difference causes the blood to begin flowing into the evacuated blood collection tubes.

Once, at 725, all of the evacuated blood collection tubes are filled, method 700 proceeds to 730, removing the hypodermic needle from the vein. The method may also include, at 735, removing the evacuated blood collection tubes from the multiport blood collection device. At 745, the method may include disposing of the multiport blood collection device following a single use.

The above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description.

What is claimed is:

1. A multiport blood collection device, comprising:
a rearward retaining portion comprising three couplers, each configured to receive a respective blood sample container;
a forward adapter portion configured to receive a connector through which a piece of tubing that is coupled to a hypodermic needle on one end is coupled to the device on the opposite end;
a branched passageway within the device to simultaneously divert blood extracted by the hypodermic needle from a piece of tubing coupled to the device through the connector inserted in the adapter portion to respective blood sample containers inserted in each of the couplers, wherein:
a first portion of the branched passageway diverts blood from the tubing to a branch point within the device, a center line of the first portion of the branched passageway being coplanar with and aligned to a center line of the adapter portion;
a second portion of the branched passageway diverts blood from the branch point to a first one of the couplers, wherein:
a center line of the second portion of the branched passageway is coplanar with the center line of the first portion of the branched passageway;
the center line of the second portion of the branched passageway is aligned to the center line of the first portion of the branched passageway and to a center line of the first coupler;
a third portion of the branched passageway diverts blood from the branch point to a second one of the couplers, wherein:
a center line of the third portion of the branched passageway is coplanar with the center line of the first portion of the branched passageway;
the center line of the third portion of the branched passageway is aligned to a center line of the second coupler;
the center line of the third portion of the branched passageway branches away from the second portion of the branched passageway on a first side of the second portion of the branched passageway at a fixed angle that is less than ninety degrees;
a fourth portion of the branched passageway diverts blood from the branch point to a third one of the couplers, wherein:
a center line of the fourth portion of the branched passageway is coplanar with the center line of the first portion of the branched passageway;
the center line of the fourth portion of the branched passageway is aligned to a center line of the third coupler;
the center line of the fourth portion of the branched passageway branches away from the second portion of the branched passageway on a second side of the second portion of the branched passageway at a fixed angle that is less than ninety degrees.

2. The multiport blood collection device of claim 1, wherein the connector comprises a female Luer taper fitting and the adapter comprises a male Luer taper fitting.

3. The multiport blood collection device of claim 1, wherein each of the couplers comprises a female Luer taper fitting.

4. The multiport blood collection device of claim 1, wherein the multiport blood collection device is comprised of a single solid body.

5. The multiport blood collection device of claim 4, wherein the multiport blood collection device is fashioned from a polycarbonate, polyetherimide, or nylon material.

6. The multiport blood collection device of claim 4, wherein the multiport blood collection device is fashioned from a material that is chemically resistant, that is transparent, and that has a smoothness between ten and twenty microns.

7. The multiport blood collection device of claim 1, wherein the multiport blood collection device is disposable.

8. The multiport blood collection device of claim 1, wherein the fixed angle at which the center line of the third portion of the branched passageway branches away from the second portion of the branched passageway and the fixed angle at which the center line of the fourth portion of the branched passageway branches away from the second portion of the branched passageway are substantially equal.

9. The multiport blood collection device of claim 8, wherein the fixed angle at which the center line of the third portion of the branched passageway branches away from the second portion of the branched passageway is about fifty degrees.

10. The multiport blood collection device of claim 1, wherein the respective distances from the branch point to each of the couplers along the second, third, and fourth portions of the branched passageways are substantially equal.

11. The multiport blood collection device of claim 1, wherein each of the blood sample containers comprises an evacuated blood collection tube within an evacuated blood collection tube holder to which a blood collection needle is coupled, the blood collection needle extending into the portion of the branched passageway that diverts blood to the coupler into which the blood sample container is inserted.

12. The multiport blood collection device of claim 1, wherein the diameters of the first, second, third, and fourth portions of the branched passageway are substantially equal.

13. The multiport blood collection device of claim 1, wherein the diameter of each of the first, second, third, and fourth portions of the branched passageway is directly proportional to an inside diameter of the hypodermic needle.

14. The multiport blood collection device of claim 13, wherein a ratio between the diameter of each of the first, second, third, and fourth portions of the branched passageway and the inside diameter of the hypodermic needle is between 3:1 and 5:1.

15. The multiport blood collection device of claim 1, further comprising:
a bracket through which a flexible strap is to be inserted, the strap comprising a fastener to attach the multiport blood collection device to an arm during use.

16. The multiport blood collection device of claim 1, wherein:
the multiport blood collection device further comprises a multi-position valve;
a portion of the branched passageway, including the branch point, is located within the multi-position valve;
the branched passageway simultaneously diverts blood from a piece of tubing coupled to the device to respective blood sample containers inserted in each of the three couplers when the multi-position valve is in a first position;
the branched passageway diverts blood from a piece of tubing coupled to the device to respective blood sample containers inserted in fewer than three couplers when the multi-position valve is in a second position;
the multi-position valve prevents blood from being diverted from a piece of tubing coupled to the three couplers when the multi-position valve is in a third position.

17. A blood collection system, comprising:
a multiport blood collection device;
a hypodermic needle;
a connector;
a piece of tubing coupled to the hypodermic needle on one end and coupled to the connector on the opposite end;
wherein the multiport blood collection device comprises:
a rearward retaining portion comprising three couplers, each configured to receive a respective blood sample container;
a forward adapter portion configured to receive the connector;
a branched passageway within the device to simultaneously divert blood extracted by the hypodermic needle from a piece of tubing coupled to the device through the connector inserted in the adapter portion to respective blood sample containers inserted in each of the couplers, wherein:
a first portion of the branched passageway diverts blood from the tubing to a branch point within the device, a center line of the first portion of the branched passageway being coplanar with and aligned to a center line of the adapter portion;
a second portion of the branched passageway diverts blood from the branch point to a first one of the couplers, wherein:
a center line of the second portion of the branched passageway is coplanar with the center line of the first portion of the branched passageway;
the center line of the second portion of the branched passageway is aligned to the center line of the first portion of the branched passageway and to a center line of the first coupler;
a third portion of the branched passageway diverts blood from the branch point to a second one of the couplers, wherein:
a center line of the third portion of the branched passageway is coplanar with the center line of the first portion of the branched passageway;
the center line of the third portion of the branched passageway is aligned to a center line of the second coupler;
the center line of the third portion of the branched passageway branches away from the second portion of the branched passageway on a first side of the second portion of the branched passageway at a fixed angle that is less than ninety degrees;
a fourth portion of the branched passageway diverts blood from the branch point to a third one of the couplers, wherein:
a center line of the fourth portion of the branched passageway is coplanar with the center line of the first portion of the branched passageway;
the center line of the fourth portion of the branched passageway is aligned to a center line of the third coupler;
the center line of the fourth portion of the branched passageway branches away from the second portion of the branched passageway on a second side of the second portion of the branched passageway at the fixed angle.

18. The blood collection system of claim 17, wherein the hypodermic needle, the connector, and the piece of tubing comprise a winged infusion set.

19. The blood collection system of claim 17, wherein:
the multiport blood collection device further comprises a multi-position valve;
a portion of the branched passageway, including the branch point, is located within the multi-position valve;
the branched passageway simultaneously diverts blood from a piece of tubing coupled to the device to respective blood sample containers inserted in each of the three couplers when the multi-position valve is in a first position;
the branched passageway diverts blood from a piece of tubing coupled to the device to respective blood sample containers inserted in fewer than three couplers when the multi-position valve is in a second position;
the multi-position valve prevents blood from being diverted from a piece of tubing coupled to the three couplers when the multi-position valve is in a third position.

20. The blood collection system of claim 17, wherein:
the multiport blood collection device further comprises a bracket;
the blood collection system further comprises a flexible strap to be inserted through the bracket, the strap comprising a fastener to attach the multiport blood collection device to an arm during use.
